(19) United States
(12) Patent Application Publication
(10) Pub. No.: US 2002/0168651 A1
(43) Pub. Date: Nov. 14, 2002

(54) Method and computer software product for determining orientation of sequence clusters

(75) Inventors: Simon Cawley, Oakland, CA (US); Raymond Wheeler, Berkeley, CA (US); Brant Wong, Walnut Creek, CA (US); Alan Williams, Albany, CA (US); David Kulp, Albany, CA (US)

Correspondence Address: AFFYMETRIX, INC, ATTN: CHIEF IP COUNSEL, LEGAL DEPT., 3380 CENTRAL EXPRESSWAY, SANTA CLARA, CA, 95051, US

(73) Assignee: AFFYMETRIX, INC., Santa Clara, CA

(21) Appl. No.: 10/006,174

(22) Filed: Dec. 4, 2001

Related U.S. Application Data
(60) Provisional application No. 60/275,456, filed on Mar. 12, 2001.

(51) Int. Cl. G01V 3/00; C12Q 1/68; G06F 19/00; G01N 33/48; G01N 33/50

(52) U.S. Cl. 435/6; 702/20

(57) ABSTRACT

In one embodiment of the invention, computerized methods are provided for determining the orientation of EST sequence Clusters. The method employs Bayesian analysis of evidence.

### RELATED APPLICATIONS

This application claims the priority of U.S. Provisional Application No. 60/275,456, Attorney Docket Number 3397, filed on Mar. 12, 2001, which is incorporated herein by reference for all purposes.

This application is related to U.S. patent application Ser. No. 09/721,042, filed on Nov. 21, 2000, entitled “Methods and Computer Software Products for Predicting Nucleic Acid Hybridization Affinity”; U.S. patent application Ser. No. 09/718,295, filed on Nov. 21, 2000, entitled “Methods and Computer Software Products for Selecting Nucleic Acid Probes” and U.S. patent application Ser. No. 09/745,965, filed on Dec. 21, 2000, entitled “Methods For Selecting Nucleic Acid Probes.” All the cited applications are incorporated herein by reference in their entireties for all purposes.

### BACKGROUND OF THE INVENTION

This invention is related to bioinformatics and biological data analysis. Specifically, this invention provides methods, computer software products and systems for determining the orientation of biological sequence clusters. In preferred embodiments, the methods, computer software products and systems are used for designing nucleic acid probe arrays.

Expressed sequence tags (ESTs) offer a rapid and relatively inexpensive way to gene discovery and functional genomics (Vasmatis et al., 1998, Discovery of three genes specifically expressed in human prostate by expressed sequence tag database analysis, Proc. Natl. Acad. Sci. USA 95(1):300-304; Adams et al., 1991, Complementary DNA sequencing : Expressed Sequence Tags and Human Genome Project. Science 252:1651-1656, both incorporated herein by reference for all purposes). EST clusters have been used for nucleic acid probe array design (U.S. Pat. No. 6,188,783, incorporated herein by reference) and for the discovery of new human genes, mapping of the human genome, and identification of coding regions in genomic sequences (Adams, et al., 1991, Science 252:1651-1656).

Because a large amount of EST data are produced using single-read sequencing in a high throughput setting, they tend to be error-prone. Various information about ESTs may be self-contradicting. For example, the orientation of EST sequences (5′-3′ direction in relation to the genes they represent) may not be simply determined according to the labels associated with the sequences. Instead, various, often contradicting evidence may have to be evaluated to determine the correct orientation. Therefore, there is a great need in the art for methods, software and systems for analyzing EST data and for using EST data for nucleic acid probe array design.

### SUMMARY OF THE INVENTION

In one aspect of the invention, computerized methods for determining whether a biological sequence has a certain characteristic are provided. The methods involve obtaining a plurality of evidence about the characteristic, where at least one evidence is sequence annotation; and determining the characteristic using a Bayesian analysis of the evidence.

In some embodiments, the step of determining includes defining a prior probability on the characteristic of the sequence; estimating the probability of the observed evidence given the value of the characteristic; and calculating the posterior probability of the characteristic given the evidence. Preferably, the step of calculating is performed according to Bayes' Rule.

In preferred embodiments, the biological sequence is a nucleic acid sequence and the characteristic is the orientation of the biological sequence. Throughout the specification, the term “biological sequence” may refer to cluster, contig or other sequence assemblies. In some particularly preferred embodiments, the nucleic acid sequence represents a cluster of nucleic acid sequences including at least one EST sequence. In some instances, the sequence is the exemplar sequence or consensus sequence of a sequence cluster. In preferred embodiments, the plurality of evidence comprises evidence from poly-A/T tail analysis, inferred splice sites; and external sequence annotation. The external sequence annotation can be, for example, CDS annotation, RNA label and EST sequence read direction.

In a particularly preferred embodiment, the method further includes testing a null hypothesis that any conflicting evidence observed is soley due to random error, against the alternative hypothesis that the observed conflicting evidence is due to systematic as opposed to random error.

In another aspect of the invention, computerized methods for designing nucleic acid probe arrays are provided. The methods include obtaining a plurality of evidence about at least one characteristic of a target nucleic acid sequence, where at least one evidence is sequence annotation; determining the characteristic using a Bayesian analysis of the evidence; and defining a target region based upon the characteristic; and selecting probes against the target region.

In some embodiments, the step of determining includes defining a prior probability that the biological sequence has the characteristic; estimating the probability of the evidence given the characteristic; and calculating the posterior probability of the characteristic given the evidence. In preferred embodiments, the step of calculating is performed according to Bayes' Rule. The characteristic of the sequence is the orientation of the target nucleic acid sequence. The target nucleic acid sequence may represent a cluster of nucleic acid sequences including at least one EST sequence. The evidence may be from poly-A/T tail analysis, inferred splice sites; and external sequence annotation which may include RNA label and EST label. The method may also include testing a null hypothesis that the orientation determination is correct and conflicting evidence observed is due solely to random error.

In another aspect of the invention, systems and computer software are provided for performing the methods of the invention. The systems include a processor; and a memory coupled with the processor, the memory storing a plurality of machine instructions that cause the processor to perform logical steps of the methods of the invention. The computer software products of the invention include a computer readable medium having computer-executable instructions for performing the methods of the invention.

### BRIEF DESCRIPTION OF THE DRAWINGS

The accompanying drawings, which are incorporated in and form a part of this specification, illustrate embodiments of the invention and, together with the description, serve to explain the principles of the invention:

FIG. 1 is a schematic showing an exemplary computer system suitable for executing some embodiments of the software of the invention.

FIG. 2 is a schematic showing the architecture of the exemplary computer system of FIG. 1.

FIG. 3 shows an exemplary computer network system suitable for executing some embodiments of the software of the invention.

FIG. 4 shows an exemplary process for using Bayesian approach to annotate a biological sequence.

### DETAILED DESCRIPTION

Reference will now be made in detail to the preferred embodiments of the invention. While the invention will be described in conjunction with the preferred embodiments, it will be understood that they are not intended to limit the invention to these embodiments. On the contrary, the invention is intended to cover alternatives, modifications and equivalents, which may be included within the spirit and scope of the invention.

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

Throughout this disclosure, various aspects of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention will employ, unless otherwise indicated, conventional techniques of bioinformatics, computer sciences, immunology, biochemistry, chemistry, molecular biology, microbiology, cell biology, genomics and recombinant DNA, which are within the skill of the art. See, e.g., Setubal and Meidanis, et al., 1997, Introduction to Computational Molecular Biology, PWS Publishing Company, Boston; Human Genome Mapping Project Resource Centre (Cambridge), 1998, Guide to Human Genome Computing, 2nd Edition, Martin J. Biship (Editor), Academic Press, San Diego; Salzberg, Searles, Kasif, (Editors), 1998, Computational Methods in Molecular Biology, Elsevier, Amsterdam; Matthews, PLANT VIROLOGY, 3^{rd} edition (1991); Sambrook, Fritsch and Maniatis, MOLECULAR CLONING: A LABORATORY MANUAL, 2^{nd} edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987))

### System for Sequence Annotation and for Nucleic Acid Probe Array Design

In aspects of the invention, methods, computer software and systems for determining the orientation of EST sequence clusters and for probe array design are provided. One of skill in the art would appreciate that many computer systems are suitable for carrying out the methods of the invention. Computer software according to the embodiments of the invention can be executed in a wide variety of computer systems.

For a description of basic computer systems and computer networks, see, e.g., Introduction to Computing Systems: From Bits and Gates to C and Beyond by Yale N. Patt, Sanjay J. Patel, 1st edition (Jan. 15, 2000) McGraw Hill Text; ISBN: 0072376902; and Introduction to Client/Server Systems : A Practical Guide for Systems Professionals by Paul E. Renaud, 2nd edition (June 1996), John Wiley & Sons; ISBN: 0471133337, both are incorporated herein by reference in their entireties for all purposes.

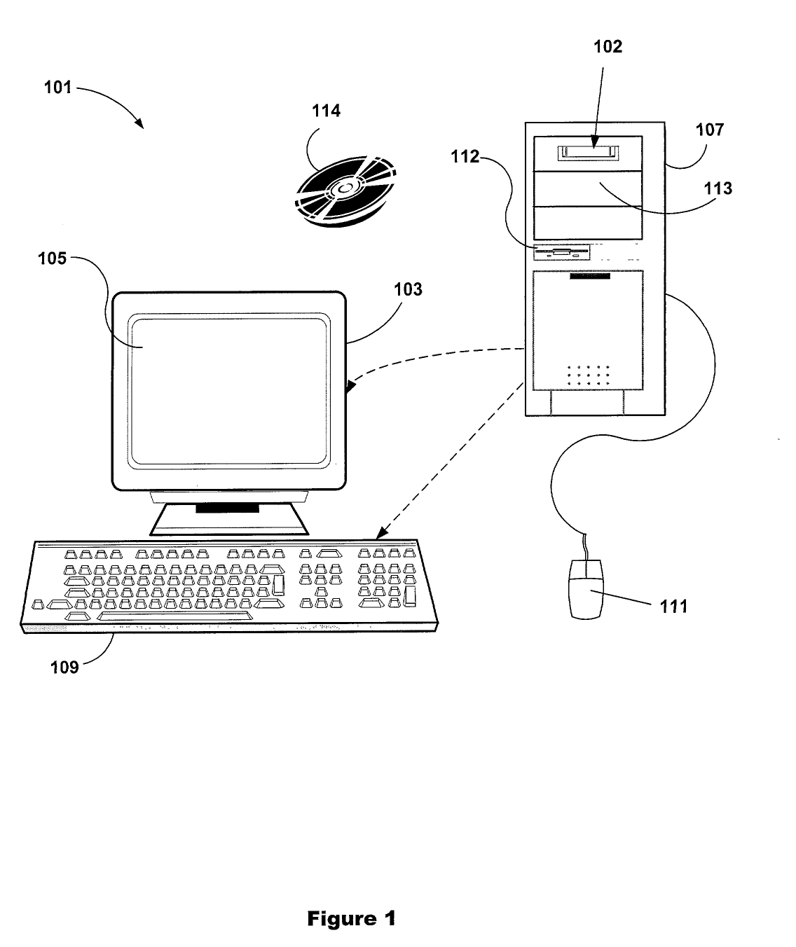

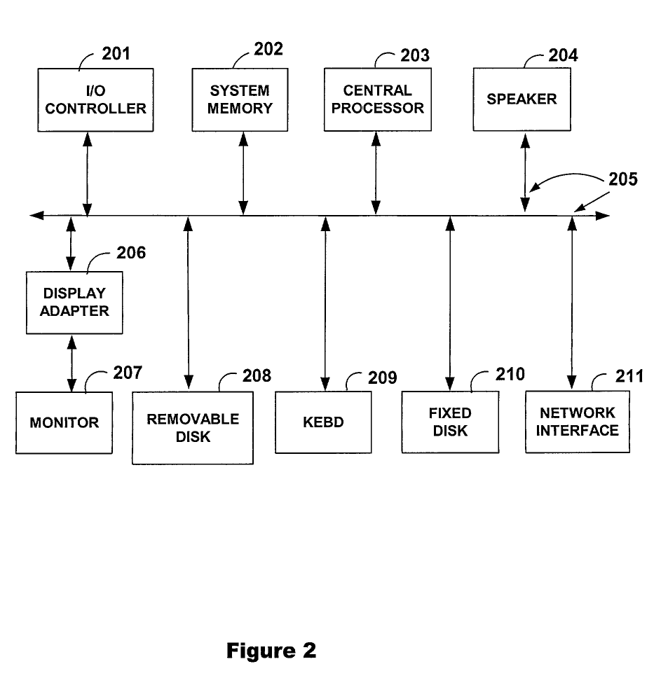

FIG. 1 illustrates an example of a computer system that may be used to execute the software of an embodiment of the invention. FIG. 1 shows a computer system 101 that includes a display 103, screen 105, cabinet 107, keyboard 109, and mouse 111. Mouse 111 may have one or more buttons for interacting with a graphic user interface. Cabinet 107 houses a floppy drive 112, CD-ROM or DVD-ROM drive 102, system memory and a hard drive (113) (see also FIG. 2) which may be utilized to store and retrieve software programs incorporating computer code that implements the invention, data for use with the invention and the like. Although a CD 114 is shown as an exemplary computer readable medium, other computer readable storage media including floppy disk, tape, flash memory, system memory, and hard drive may be utilized. Additionally, a data signal embodied in a carrier wave (e.g., in a network including the Internet) may be the computer readable storage medium.

FIG. 2 shows a system block diagram of computer system 101 used to execute the software of an embodiment of the invention. As in FIG. 1, computer system 101 includes monitor 201, and keyboard 209. Computer system 101 further includes subsystems such as a central processor 203 (such as a Pentium™ III processor from Intel), system memory 202, fixed storage 210 (e.g., hard drive), removable storage 208 (e.g., floppy or CD-ROM), display adapter 206, speakers 204, and network interface 211. Other computer systems suitable for use with the invention may include additional or fewer subsystems. For example, another computer system may include more than one processor 203 or a cache memory. Computer systems suitable for use with the invention may also be embedded in a measurement instrument.

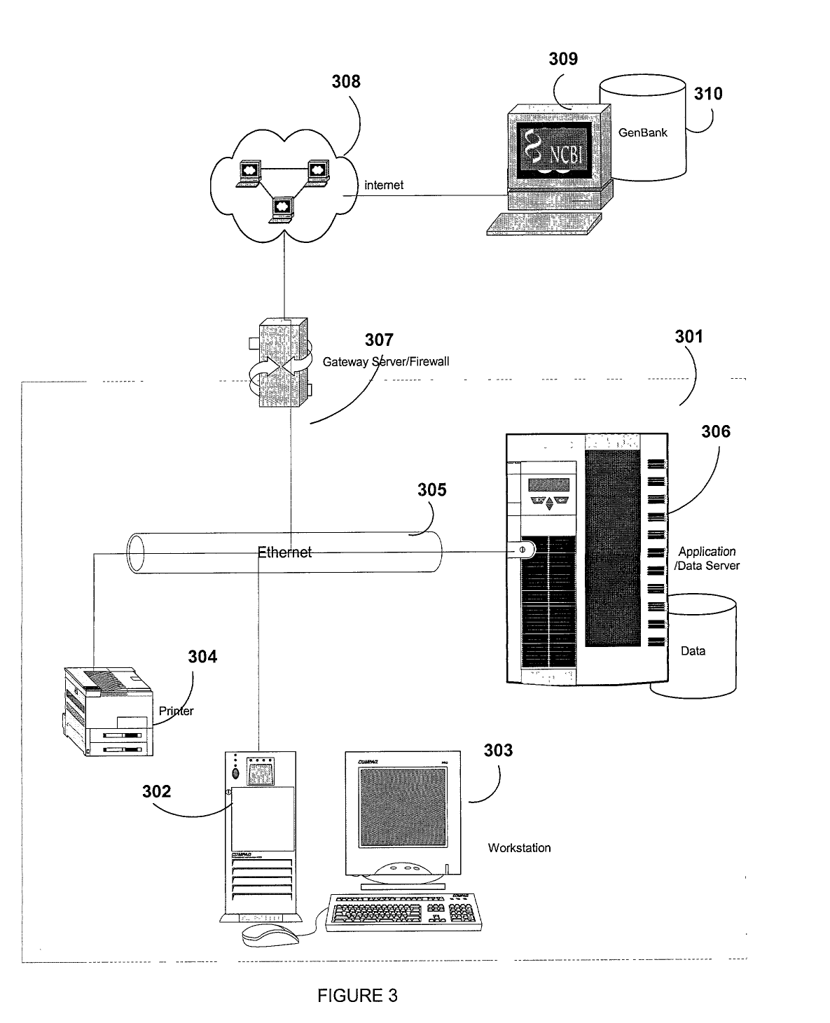

FIG. 3 shows an exemplary computer network that is suitable for executing the computer software of the invention. A computer workstation 302 is connected with the application/data server(s) through a local area network (LAN) 301, such as an Ethernet 305. A printer 304 may be connected directly to the workstation or to the Ethernet 305. The LAN may be connected to a wide area network (WAN), such as the Internet 308, via a gateway server 307 which may also serve as a firewall between the WAN 308 and the LAN 305. In preferred embodiments, the workstation may communicate with outside data sources, such as the National Biotechnology Information Center, through the Internet. Various protocols, such as FTP and HTTP, may be used for data communication between the workstation and the outside data sources. Outside genetic data sources, such as the GenBank 310, are well known to those skilled in the art. An overview of GenBank and the National Center for Biotechnology information (NCBI) can be found in the web site of NCBI (“www.ncbi.nlm.nih.gov”).

Computer software products of the invention typically include computer readable medium having computer-executable instructions for performing the logic steps of the methods of the invention. Suitable computer readable medium include floppy disk, CD-ROM/DVD/DVD-ROM, hard-disk drive, flash memory, ROM/RAM, magnetic tapes and etc. The computer executable instructions may be written in any suitable computer language or combination of several languages. Suitable computer languages include C/C++ (such as Visual C/C++), C#, Java, Basic (such as Visual Basic), SQL, Fortran, SAS and Perl.

Nucleic Acid Probe Arrays

The methods, computer software and systems of the invention are particularly useful for designing high density nucleic acid probe arrays.

High density nucleic acid probe arrays, also referred to as “DNA Microarrays,” have become a method of choice for monitoring the expression of a large number of genes and for detecting sequence variations, mutations and polymorphism. As used herein, “nucleic acids” may include any polymer or oligomer of nucleosides or nucleotides (polynucleotides or oligonucleotidies), which include pyrimidine and purine bases, preferably cytosine, thymine, and uracil, and adenine and guanine, respectively. See Albert L. Lehninger, PRINCIPLES OF BIOCHEMISTRY, at 793-800 (Worth Pub. 1982) and L. Stryer, BIOCHEMISTRY, 4^{th} Ed. (March 1995), both incorporated by reference. “Nucleic acids” may include any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

“A target molecule” refers to a biological molecule of interest. The biological molecule of interest can be a ligand, receptor, peptide, nucleic acid (oligonucleotide or polynucleotide of RNA or DNA), or any other of the biological molecules listed in U.S. Pat. No. 5,445,934 at col. 5, line 66 to col. 7, line 51, which is incorporated herein by reference for all purposes. For example, if transcripts of genes are the interest of an experiment, the target molecules would be the transcripts. Other examples include protein fragments, small molecules, etc. “Target nucleic acid” refers to a nucleic acid (often derived from a biological sample) of interest. Frequently, a target molecule is detected using one or more probes. As used herein, a “probe” is a molecule for detecting a target molecule. It can be any of the molecules in the same classes as the target referred to above. A probe may refer to a nucleic acid, such as an oligonucleotide, capable of binding to a target nucleic acid of complementary sequence through one or more types of chemical bonds, usually through complementary base pairing, usually through hydrogen bond formation. As used herein, a probe may include natural (i.e. A, G, U, C, or T) or modified bases (7-deazaguanosine, inosine, etc.). In addition, the bases in probes may be joined by a linkage other than a phosphodiester bond, so long as the bond does not interfere with hybridization. Thus, probes may be peptide nucleic acids in which the constituent bases are joined by peptide bonds rather than phosphodiester linkages. Other examples of probes include antibodies used to detect peptides or other molecules, any ligands for detecting its binding partners. When referring to targets or probes as nucleic acids, it should be understood that these are illustrative embodiments that are not to limit the invention in any way.

In preferred embodiments, probes may be immobilized on substrates to create an array. An “array” may comprise a solid support with peptide or nucleic acid or other molecular probes attached to the support. Arrays typically comprise a plurality of different nucleic acids or peptide probes that are coupled to a surface of a substrate in different, known locations. These arrays, also described as “microarrays” or colloquially “chips” have been generally described in the art, for example, in Fodor et al., Science, 251:767-777 (1991), which is incorporated by reference for all purposes. Methods of forming high density arrays of oligonucleotides, peptides and other polymer sequences with a minimal number of synthetic steps are disclosed in, for example, U.S. Pat. Nos. 5,143,854, 5,252,743, 5,384,261, 5,405,783, 5,424,186, 5,429,807, 5,445,943, 5,510,270, 5,677,195, 5,571,639, 6,040,138, all incorporated herein by reference for all purposes. The oligonucleotide analogue array can be synthesized on a solid substrate by a variety of methods, including, but not limited to, light-directed chemical coupling, and mechanically directed coupling. See Pirrung et al., U.S. Pat. No. 5,143,854 (see also PCT Application No. WO 90/15070) and Fodor et al., PCT Publication Nos. WO 92/10092 and WO 93/09668, U.S. Pat. Nos. 5,677,195, 5,800,992 and 6,156,501, which disclose methods of forming vast arrays of peptides, oligonucleotides and other molecules using, for example, light-directed synthesis techniques. See also, Fodor, et al., Science, 251, 767-77 (1991). These procedures for synthesis of polymer arrays are now referred to as VLSIPS™ procedures.

Methods for making and using molecular probe arrays, particularly nucleic acid probe arrays are also disclosed in, for example, U.S. Pat. Nos. 5,143,854, 5,242,974, 5,252,743, 5,324,633, 5,384,261, 5,405,783, 5,409,810, 5,412,087, 5,424,186, 5,429,807, 5,445,934, 5,451,683, 5,482,867, 5,489,678, 5,491,074, 5,510,270, 5,527,681, 5,527,681, 5,541,061, 5,550,215, 5,554,501, 5,556,752, 5,556,961, 5,571,639, 5,583,211, 5,593,839, 5,599,695, 5,607,832, 5,624,711, 5,677,195, 5,744,101, 5,744,305, 5,753,788, 5,770,456, 5,770,722, 5,831,070, 5,856,101, 5,885,837, 5,889,165, 5,919,523, 5,922,591, 5,925,517, 5,658,734, 6,022,963, 6,150,147, 6,147,205, 6,153,743 and 6,140,044, all of which are incorporated by reference in their entireties for all purposes.

Microarray can be used in a variety of ways. A preferred microarray contains nucleic acids and is used to analyze nucleic acid samples. Typically, a nucleic acid sample is prepared from appropriate source and labeled with a signal moiety, such as a fluorescent label. The sample is hybridized with the array under appropriate conditions. The arrays are washed or otherwise processed to remove non-hybridized sample nucleic acids. The hybridization is then evaluated by detecting the distribution of the label on the chip. The distribution of label may be detected by scanning the arrays to determine fluorescence intensity distribution. Typically, the hybridization of each probe is reflected by several pixel intensities. The raw intensity data may be stored in a gray scale pixel intensity file. The GATC™ Consortium has specified several file formats for storing array intensity data. The final software specification is available at www.gatcconsortium.org and is incorporated herein by reference in its entirety. The pixel intensity files are usually large. For example, a GATC™ compatible image file may be approximately 50 Mb if there are about 5000 pixels on each of the horizontal and vertical axes and if a two byte integer is used for every pixel intensity. The pixels may be grouped into cells (see, GATC™ software specification). The probes in a cell are designed to have the same sequence (i.e., each cell is a probe area). A CEL file contains the statistics of a cell, e.g., the 75th percentile and standard deviation of intensities of pixels in a cell. The 50, 60, 70, 75 or 80th percentile of pixel intensity of a cell is often used as the intensity of the cell.

Nucleic acid probe arrays have found wide applications in gene expression monitoring, genotyping and mutation detection. For example, massive parallel gene expression monitoring methods using nucleic acid array technology have been developed to monitor the expression of a large number of genes (e.g., U.S. Pat. Nos. 5,871,928, 5,800,992 and 6,040,138; de Saizieu et al., 1998, Bacteria Transcript Imaging by Hybridization of total RNA to Oligonucleotide Arrays, NATURE BIOTECHNOLOGY, 16:45-48; Wodicka et al., 1997, Genome-wide Expression Monitoring in Saccharomyces cerevisiae, NATURE BIOTECHNOLOGY 15:1359-1367; Lockhart et al., 1996, Expression Monitoring by Hybridization to High Density Oligonucleotide Arrays. NATURE BIOTECHNOLOGY 14:1675-1680; Lander, 1999, Array of Hope, NATURE-GENETICS, 21(suppl.), at 3, all incorporated herein by reference for all purposes). Hybridization-based methodologies for high throughput mutational analysis using high-density oligonucleotide arrays (DNA chips) have been developed, see Hacia et al., 1996, Detection of heterozygous mutations in BRCA1 using high density oligonucleotide arrays and two-color fluorescence analysis. Nat. Genet. 14:441-447, Hacia et al., New approaches to BRCA1 mutation detection, Breast Disease 10:45-59 and Ramsey 1998, DNA chips: State-of-Art, Nat Biotechnol. 16:40-44, all incorporated herein by reference for all purposes). Oligonucleotide arrays have been used to screen for sequence variations in, for example, the CFTR gene (U.S. Pat. No. 6,027,880, Cronin et al., 1996, Cystic fibrosis mutation detection by hybridization to light-generated DNA probe arrays. Hum. Mut. 7:244-255, both incorporated by reference in their entireties), the human immunodeficiency virus (HIV-1) reverse transcriptase and protease genes (U.S. Pat. No. 5,862,242 and Kozal et al., 1996, Extensive polymorphisms observed in HIV-1 clade B protease gene using high density oligonucleotide arrays. Nature Med. 1:735-759, both incorporated herein by reference for all purposes), the mitochondrial genome (Chee et al., 1996, Accessing genetic information with high density DNA arrays. Science 274:610-614) and the BRCA1 gene (U.S. Pat. No. 6,013,449, incorporated herein by reference for all purposes).

Methods for signal detection and processing of intensity data are additionally disclosed in, for example, U.S. Pat. Nos. 5,445,934, 547,839, 5,578,832, 5,631,734, 5,800,992, 5,856,092, 5,936,324, 5,981,956, 6,025,601, 6,090,555, 6,141,096, 6,141,096, and 5,902,723. Methods for array based assays, computer software for data analysis and applications are additionally disclosed in, e.g., U.S. Pat. Nos. 5,527,670, 5,527,676, 5,545,531, 5,622,829, 5,631,128, 5,639,423, 5,646,039, 5,650,268, 5,654,155, 5,674,742, 5,710,000, 5,733,729, 5,795,716, 5,814,450, 5,821,328, 5,824,477, 5,834,252, 5,834,758, 5,837,832, 5,843,655, 5,856,086, 5,856,104, 5,856,174, 5,858,659, 5,861,242, 5,869,244, 5,871,928, 5,874,219, 5,902,723, 5,925,525, 5,928,905, 5,935,793, 5,945,334, 5,959,098, 5,968,730, 5,968,740, 5,974,164, 5,981,174, 5,981,185, 5,985,651, 6,013,440, 6,013,449, 6,020,135, 6,027,880, 6,027,894, 6,033,850, 6,033,860, 6,037,124, 6,040,138, 6,040,193, 6,043,080, 6,045,996, 6,050,719, 6,066,454, 6,083,697, 6,114,116, 6,114,122, 6,121,048, 6,124,102, 6,130,046, 6,132,580, 6,132,996 and 6,136,269, all of which are incorporated by reference in their entireties for all purposes.

Nucleic Acid Probe Array Design Process

In some embodiments, a nucleic acid probe array design process involves selecting the target sequences and selecting probes. For example, if the probe array is designed to detect the expression of genes at the transcript level. The target sequences are typically transcript sequences. Selection of the target sequence may involve the characterization of the target sequence based upon available information. For example, expressed sequence tags information needs to be assembled and annotated.

After target sequences are identified, probes for detecting the target sequences can be selected. The probe sequences and layout information are then translated to photolithographic masks, commands for controlling ink-jet directed synthesis, or soft lithographic synthesis process.

EST Clusters and Evidence for Their Directions

Nucleic acid probe array design, particular for arrays detecting gene expression, often involves the analysis of “EST”, or “Expressed Sequence Tag”. EST as used herein, refers to a fragment of a cDNA clone that has been at least partially sequenced. For a detailed discussion of the process of producing ESTs, see. e.g., Baldo et al., 1996, Normalization and Substraction: Two Approaches to Facilitate Gene Discovery. Genome Research 6:791-806, which is incorporated herein by reference for all purposes.

With the easy access to technology to generate ESTs, tens of thousands of ESTs are sequenced. The high volume and high throughput nature of the EST data often results in high error rates (Aaronson et al., 1996, et al., Toward the Development of a Gene Index to the Human Genome: An Assessment of the Nature of High Throughput EST sequence Data. Genome Research 6:829-845, incorporated herein by reference for all purposes). Typically, a single read is generated for each EST. Errors typically include wrong clone orientation, associated clone ID chimeras, missing 3′ and 5′ reads.

A common way to assemble ESTs is by clustering. The goal of such a project is the construction of a gene index in which ESTs and full-length transcripts are partitioned into index classes (or clusters) such that they are placed in the same index class if and only if they represent the same gene. Projects related to EST clustering and assembly include UniGene from the National Center for Biotechnology Information; the TIGR Gene Index (http://www.tigr.org/tdb/hgi/hgi.html) from the Institute for Genomic Research; the Sequence Tag Alignment and Consensus Knowledgebase (STACK; bttp://ziggy.sanbi.ac.za/stack/stacksearch.htm); the Merck/Washington University Gene Index; and the GenExpress project. All of these projects perform some type of cluster analysis in which sequence similarity is used to form the clusters. For an overview of EST clustering, see, Win Hide and Alan Christoffels, EST Clustering Tutorial, ISMB, 1999 (available at www.sanbi.ac.za) and incorporated here by reference. It is worth noting that the gene indexing process typically incorporate information about EST and full length cDNA sequences.

Directional Call Using Bayesian Approach

Typically, the direction of a cluster is indicated by the following evidence: 1) external sequence annotation may include direction information; 2) poly A/T tail analysis may provide directional information; and 3) inferred splice sites also provide directional information. Unfortunately, the evidence may not point to a consistent direction.

The inconsistency of evidence may result because mislabeling of ESTs. 5′ EST clones may be mistakenly labeled as 3′EST clones by error, and vice versa; Clone reversal; Chimeric clones, sequence reverse complementation and etc.

U.S. patent application Ser. No. 09/764,324, which is incorporated herein by reference in its entirety for all purposes, provide a statistical method for resolving orientation (5′->3′) of sequence clusters. However, additional methods are still needed.

In one aspect of the invention, a Bayesian approach is used to determine the orientation of sequence clusters or subclusters, which has the advantage of an underlying probabilistic structure allowing for natural and interpretable means of incorporating different sources of evidence into the call. Moreover, it allows the use of prior knowledge of such as the probability of EST labels or consensus directions.

The Bayesian approach of the invention is particularly useful for determining the orientation of sequence clusters. However, the application of the approach is not limited to determining the orientation of sequence clusters. Rather, this invention is useful for characterizing many aspects of a biological sequence using a variety of evidence, particularly when the evidence is not conclusive and may contain erroneous information, such as labeling errors.

The Bayesian approach for updating belief in a statement S (i.e. the probability of S) given additional evidence E, and background information (context) I is estimated according to the Bayes Rule: p(S|E,I)=p(S|I)*p(E|S,I)/p(E|I). The left-hand term, p(S|E,I), is called the posterior probability, and it is the probability of S after considering the effect of evidence E in context I. The p(S|I) term is the prior probability of S given I alone; that is, the belief in S before the evidence E is considered.

p(E|S,I) is the likelihood, or the probability of the evidence assuming statement S and background information I is true. The last term, 1/p(E|I), is independent of H, and can be regarded as a normalizing or scaling constant. The information I is a conjunction of (at least) all of the other statements relevant to determining p(S|I) and p(E|I).

Because all probability is conditional, as each new piece of evidence is factored into the calculation, its effect is conditional on all the previously considered evidence. This effect may be overlooked by making conditional independence assumptions, such as: p(E2|E1,I)=p(E2|I) and p(E1|E2,I)=p(E1|I).

In other words: given I, knowing that E2 is true tells us nothing about E1, and vice versa. Thus E2 contains no information regarding E1 that is not already present in I. Under conditional independence the product rule reduces to: p(E1,E2|I)=p(E1|I)*p(E2|I).

And when multiple evidence Ei are conditionally independent under I, and thus S,I, the multiple update version of Bayes' rule reduces to: p(S|I E1 E2 E3 . . . )=(p(S|I)*p(E1|S,I)*p(E2|S,I)*p(E3|S,I) . . . )/(p(E1|I)*p(E2|I)*p(E3|I) . . . ). One of skill in the art would appreciate that while this equation greatly simplifies the problem of incorporating evidence, it may not be useful for sequence analysis in cases while the conditional independence assumption is not true.

For a description of the Bayesian approach and its application to data analysis, see, e.g., Bayesian Data Analysis, by Andrew Gelman, John B. Carlin, Hal S. Stem, Donald B. Rubin, 1995, CRC Press; ISBN: 0412039915; Bayesian Theory (Wiley Series in Probability and Statistics) by Jose M. Bernardo, Adrian F. M. Smith, 2001, John Wiley & Sons, 2001, ISBN: 047149464X; Data Analysis : A Bayesian Tutorial (Oxford Science Publications) by D. S. Sivia, 1996, Oxford University Press, ISBN: 0198518897.

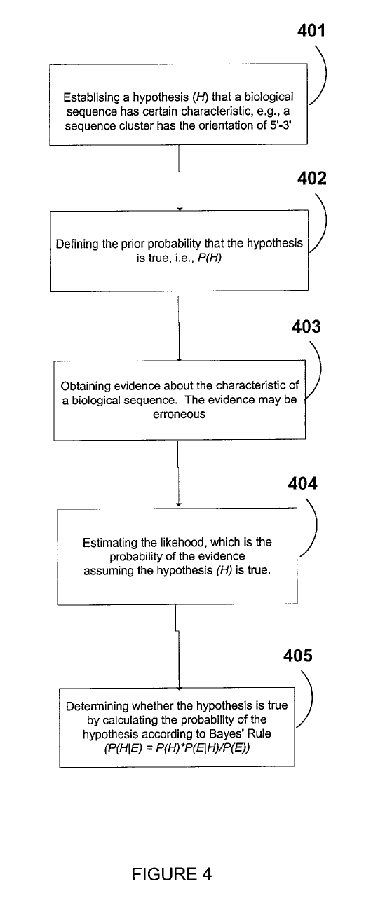

FIG. 4 shows a computerized process for determining a characteristic of a biological sequence based upon evidence. A statement (S) that a biological sequence has certain characteristic, e.g., a sequence cluster has the orientation of 5′-3′, is used (401). A prior probability that the statement is true, i.e., P(S) is given (e.g., by user input into a computer system). The prior probability may be somewhat subjective and is given before the evidence is considered. In a typical embodiment, the prior probability is the overall probability that the biological sequence has certain characteristic. Evidence about the characteristic of a biological sequence is obtained into the computer system by any suitable means such as a file, a data communication stream, etc (403). The process of the invention is particularly useful for processing evidences that may not be perfectly reliable. The likelihood that the statement S is true is computed (404) and the probability that the statement S is true is determined according to the Bayes' Rule (405).

The process is particularly suitable for analyzing the orientation of sequence clusters that contains EST sequences and will be illustrated using this particular sequence annotation problem as an example.

Throughout the specification and the claims, certain notations are used for the purpose of better describing embodiments of the invention. These notations are arbitrarily chosen. One of skill in the art would appreciate that the scope of the invention is NOT limited to the particular set of notations. Table 1 lists some of the notations used throughout the specification.

TABLE 1
Notations

| Notation | Meaning | Value |
|---|---|---|
| c | consensus direction | {+, −}, |
| | | one of the two directions |
| aᵢ | alignment strand of every | {+, −} |
| | sequence in the cluster | |
| rᵢ | labeled strandedness for | {+, −} |
| | RNAs | |
| lᵢ | labeled strandedness for | {+, −} |
| | ESTs | |
| sᵢ | splicing-indicated strand for | {+, −} |
| | all sequences | |
| dᵢ | Polyadenylation indicated | {+, −} |
| | strand for all sequences | |

In preferred embodiments, methods are provided to compute P(c=+|a,r,l,s,d). Here, a loose notation is used, i.e., a is used represent (a₁, . . . , aₙ) and so on. According to Bayes' law, P(c=+|a,r,l,s,d)=P(a,r,l,s,d|c=+) * P(c=+)/P(a,r,l,s,d) and the numerator can be expressed as: P(a,r,l,s,d)=P(a,r,l,s,d|c=+)P(c=+)+P(a,r,l,s,d|c=−)P(c=−).

P(c=+) could in principle be specified in advance—for example if it is known that the sub-clustering procedure is 80% likely to produce an assembly with the consensus in the sense orientation (e.g., because of long mRNAs being entered in the sense orientation and a clustering procedure tending to favor assembling longer sequences into the sense orientation) then P(c=+) could be set to, e.g., 0.6, 0.7, 0.8 or 0.9. In some embodiments, no bias is assumed and P(c=+) is set to be 0.5.

Given P(c=+), P(a,r,l,s,d|c=+) and P(a,r,l,s,d|c=−) can be computed: P(a,r,l,s,d|c=+)=P(a|c=+) P(r|a,c=+) P(l|a,c=+) P(s|a,l,c=+) P(d|a,l,c=+). In the above example, a few assumptions are made to simplify the calculation. Firstly, it is assumed that the EST-related evidence (EST labelling, polyadenyllation and splicing) is independent of the RNA evidence, which keeps the r term out of the last three conditional probabilities. This should be quite a reasonable assumption. Secondly, it is assumed that P(d|s,a,l,c=+) is independent of s, or in other words that Polyadenylation evidence is independent of splicing evidence. The individual parts in the above can be computed as follows:

P(a|c=+): In some preferred embodiments, because ESTs are more likely to be 3′ than 5|, this can be set such that p(a=−|c=+) is larger than 0.5. In some other embodiments, however, an unbiased approach is taken and this is set to 0.5.

P(l|a,c): is given by taking the product over the following conditional probability distribution, in which 1blerr is the probability of a mislabelling in an EST. For this step we give ESTs a high error probability of at least 10, 15, 20, or 30%, reducing their effect in the calling of direction when other kinds of evidence are present. The same applies to the calculation of P(r|a,c), with a different value of 1blerr.

TABLE 2
Calculation of P(l|a,c)

| P(l = +) | P(l = −) | A | C |
|---|---|---|---|
| l − lblerr | lblerr | + | + |
| lblerr | l − lblerr | − | + |
| lblerr | l − lblerr | + | − |
| l − lblerr | lblerr | − | − |

P(d|l,a,c): is computed taking products from the following conditional probability table (Table 3). P(d) is the probability that the polyadenylation evidence is correct. In one particularly preferred embodiment, on the first pass P(d) is set to the heuristic score returned by a polyadenylation scoring module. After assessment of the initial results, an empirical estimate of P(d) is producted. m is an extra factor which is added/subtracted from the score according to whether the labelling agrees or disagrees with the polyA evidence—arbitrarily set to 0.05 in some embodiments. (Values outside the range (0,1) are truncated to be within the range).

TABLE 3
Calculation of P(d|l,a,c)

| P(d = +) | P(d = −) | L | A | c |
|---|---|---|---|---|
| P(d) + m | l − (P(d) − m) | + | + | + |
| P(d) − m | l − (P(d) + m) | − | + | + |
| l − (P(d) + m) | P(d) − m | + | − | + |
| l − (P(d) − m) | P(d) + m | − | − | + |
| l − (P(d) + m) | P(d) − m | + | + | − |
| l − (P(d) − m) | P(d) + m | − | + | − |
| P(d) + m | l − (P(d) − m) | + | − | − |
| P(d) − m | l − (P(d) + m) | − | − | − |

P(s|l,a,c) is computed similarly to P(d|l,a,c), according to the following probability table. In what follows, P(s) is the score of the splicing evidence. In some embodiments, on the first pass an arbitrary value of 0.95 is used and thereafter an empirical estimate is used. m is the adjustment for agreement/disagreement with the labeling.

TABLE 4
Calculation of P(s|l,a,c)

| P(s = +) | P(s = −) | l | a | c |
|---|---|---|---|---|
| P(s) + m | l − (P(s) − m) | + | + | + |
| P(s) − m | l − (P(s) + m) | − | + | + |
| l − (P(s) + m) | P(s) − m | + | − | + |
| l − (P(s) − m) | P(s) + m | − | − | + |
| l − (P(s) + m) | P(s) − m | + | + | − |
| l − (P(s) − m) | P(s) + m | − | + | − |
| P(s) + m | l − (P(s) − m) | + | − | − |
| P(s) − m | l − (P(s) + m) | − | − | − |

The model just outlined allows for the calculation of P(c=+|l,a,s,p) and P(c=−|l,a,s,p), the consensus is called as the higher of the two values. In the case of a tie the arbitrary call of + is used in some preferred embodiment.

The Bayesian approach is particulary useful for analyzing the orientation of EST or other sequence clusters. In addition, this approach is also useful for other aspects of gene characterization where multiple inconsistent evidence need to be considered. For example, the Bayesian approach may be used to analyze the coding content or the hybridization affinity of a sequence.

Cluster Quality Checking

After generating a tentative estimate of the orientation of sequence clusters, any evidence which conflicts with the call may be considered. In some embodiments, for each of four evidence types (RNA label, EST label, polyadenylation and splicing), the following is recorded: x, the number of sequences which conflict with the estimated orientation; n, the number of sequences of the evidence type.

A null hypothesis that the estimated orientation is correct, and that any conflicting evidence observed is due to random as opposed to systematic error is tested. The model is that x is a realization from a binomial process with error rate p (which will depend on the evidence type in consideration) and with sample size n. The alternative hypothesis is that the error rate is larger than p, a one-tailed hypothesis test is performed. If n*p is large enough (greater than about 5), normal approximation can be used, i.e., a z-statistic (x−np)/(sqrt(np(1−p)) can be computed and the p-value of the test is the area under the standard normal curve above this z-value. If n*p is small the normal approximation doesn't hold, in which case an exact binomial test may be used, the p-value is Sum {k=x}ˆ n (n choose k) pˆ k(1−p)ˆ {n−k}.

Given a confidence level alpha, the null hypothesis can be rejected if the p-value is less than alpha. In some embodiment, the hypothesis test is performed for each evidence type, if the null is rejected for any then the subcluster is declared to be problematic. In such a case the cluster is flagged and two lists are made, each is a list of the sequences suggesting a particular orientation for the consensus. These lists form new inputs to a re-subclustering process.

If the null hypothesis is not rejected for each evidence type, any observed conflicts are explainable by random errors. As a final pass, we set a threshold T such that if the posterior probability of the estimated orientation is less than T the sequence orientation is reclassified as ‘U’ or unknown. The higher T, the more conservative the calling procedure.

The final thing to consider is what values of the error probability p and what hypothesis test significance level to use for the different evidence types. Since we place very high confidence on RNA data, if there are any conflicts at all in the RNA labeling evidence we declare the cluster to be problematic. For the other evidence types we set the error probability to be one minus the empirical estimate of the probability for the evidence type (as mentioned above). We propose tesing at a significance level of 0.01. In statistical terms this means that the probability of type I error for the hypothesis tests is 1%. In other words, we set the “false alarm” rate to 1%.

In yet another aspect of the invention, the method is used to analyze the orientation of a single sequence as a special case (treating it as a cluster of only one member). A call can be made first at the level of each individual sequence. Such individual sequence calls can then be combined later on at the cluster level.

Example: Application to DNA Probe Array Design

The following example shows an embodiment of the invention with application to nucleic acid probe array design.

A. Data Collection. UniGene (based on BLAST similarity) sequence data were used to serve as the basis of clustering. Sequence information from non-Unigene sources, GenBank, RefSeq, and dbEST were also used. Golden Path data [citation] served as a quality control of the sequence information.

UniGene: UniGene files Hs.data and Hs.seq.all were downloaded. Hs.data is the clustering information file and Hs.seq.all is the sequence information file for Hs.data.

GenBank: GenBank records were parsed for human mRNA records and divided into two sets. Records containing the words “complete cds” in the definition line were grouped into the “cmrna” set. All other human records were grouped into the “mrna” set.

RefSeq: RefSeq records were parsed for human mRNA records and grouped into one set called the “rsmrna” set.

dbEST: dbEST records were parsed for human records and grouped into one set called the “dbEST” set.

GenBank DNA records: There are several UniGene references to DNA records in GenBank. These sequences from UniGene (Hs.seq.all) were extracted. This set is called the “dmrna” set.

Unlinkable references: There were some UniGene references that could not be found in the data sets (i.e. GSS sequences). These sequences were manually assessed for inclusion and then extracted from UniGene (Hs.seq.all). This set is called the “manual” set.

The sets cmrna and rsmrna represent the set of full-length sequences. The set of mrna, cmrna, and rsmrna represent the set of known genes.

B. Genomic Mapping and Assessment. All the sequences were mapped to the public human genome sequence using psLayout. This serves to verify the validity of the orientations using consensus splice sites, detect chimeric UniGene clusters, determine dbEST genomic trimming, and allows us to give more information to the user in terms of location in the genome. The orientation of a sequence can be determined if the sequence spans an intron and by looking at the sequence content on both sides of the splice site. Chimeric clusters can be detected if the cluster maps well to two different locations in the genome. dbEST trimming can be aided here by flagging and annotating low quality sequence if one end of the EST aligns very well and the other end aligns poorly. Additional user information is inherent to the genome location information of the mappings

C. Contamination Screening and Masking. Vector screening was performed using the UniVector database (NCBI) and BLAST. Screening of repeats and other low quality regions is performed using repeatMasker. This was performed so that unwanted sequence data does not contaminate the probes. Significant repeats and low complexity sequences are masked (masked base is replaced by an “N”).

D. Cluster Creation. Clustering information was taken from UniGene (Hs.data). This file details which sequences belong in which cluster and the sequences are distributed accordingly, each cluster having its own directory.

E. dbEST Alignments and Trimming. Because EST sequence quality can vary greatly between sequences as well as within sequences, the final EST sequences that were used for probe array design must be known to be of good quality. These were the steps used to trim dbEST (the next step is performed only if the previous step failed): Trim according to sequence quality annotation in dbEST report files; Submissions from “The Institute for Genomic Research” and “Genethon” are considered already trimmed; Sequences with Wash-U quality scores are considered already trimmed; Trim according to genomic alignment with at least 90% identity across the entire EST or the alignment block of at least 180 bases; Sequences from groups submitting less than 100 sequences are considered trimmed; trim from the right side to the mean high quality length for submissions in that year; and Sequences before 1995 have been considered pre-trimmed.

Subclustering. Subclustering was performed to generate consensus sequences for each cluster using CAT. Additional subclustering is performed based on genomic alignments of the member sequences and the orientation assessment of the cluster. Genomic alignments are performed before running CAT, and orientation assessments are performed after running CAT. To reduce the compute time, clusters with more than 500 members were pruned. If there were still more than 500 sequences, additional manual pruning occurred down to 500 members. We found CAT to have difficulties with datasets larger than this. The CAT runs were executed in sets according to the number of sequences in the cluster. The sets were 2-100, 101-500, and >500 (pruned). Clusters with only one sequence were instead processed by a script that generated clusters simulating the CAT-converted cluster tree.

G. False Priming. The false priming process annotates sequences that may incorrectly indicate an alternative poly-adenylation site when there is a poly-A or poly-T region in the genome. If the poly-A/T tail and the downstream region aligns to the genome, the sequence is considered to be falsely primed and is not used as evidence for a poly-adenylation site.

H. Orientation. Orientation determination was used to assess the directionality of each subcluster consensus sequence. This is important because of the way transcripts are amplified. This step also determines whether a subcluster needs to be resubclustered to clear any conflicts in orientation evidence. Orientation determination takes three forms: Poly-A/T tail analysis; inferred splice sites; and external sequence annotation.

An orientation call on a cluster can be one of four things: sense, anti-sense, unknown, or problematic. If the sequences in the subcluster score highly toward sense or anti-sense, it is called appropriately. If there is no information from any of the orientation methods, it is labeled as unknown. Unknown sequences will be tiled from both ends. If the sequence is labeled problematic, the cluster is resubclustered.

For every sub-cluster, a call is made as to whether it is: sense, anti-sense, unknown, or problematic. Problematic subclusters were flagged and split into two sets, sequences aligning with the forward strand and those aligning with the reverse. Each set was submitted to reclustering. This led to multiple sub-clusters for each of the two sets, all of which then have to be run through the Orientation step. The Bayesian approach discussed above was used to make the directional call.

First, the orientation each sequence implies for the cluster was determined. In each cluster, sequence files were converted into evidence files for use in the orientation calling step. In the case of ESTs, a hash keyed by GI which points to WUSTL traces where available was used.

Adding Unincluded Full-lengths. Remaining full-length sequences that had not been included in the pipeline thus far were added as additional sequences to select probes. These are not clustered with any other sequences.

J. Consensus Calling and Alignment. Consensus sequence were determined according to methods disclosed in U.S. application Ser. No. ______.

K. Final Sequence Selection. For each consensus sequence the region 5′ of each poly-adenylation cluster were chosen for probe selection. A poly-adenylation cluster is a small region (˜30 bp) containing one or more poly-adenylation sites. The region chosen for probe selection did not extend 5′ beyond the lesser of the next poly-adenylation site or 600 bases. 5′ poly-adenylation sites tended to be favored, biologically, over 3′ poly-adenylation sites. This sequence selection strategy had been designed to capture this phenomenon.

In addition to computationally identified poly-adenylation sites, the 3′ ends of all full-length mRNAs were considered a poly-adenylation site. If a poly-adenylation cluster contained a full-length sequence, then a subsequence would be choosen from the full-length sequence (rather than the consensus sequence) and that subsequence would extend the full 600 bp upstream regardless of any upstream poly-adenylation clusters.

All publications and patent applications cited above are incorporated by reference in their entirety for all purposes to the same extent as if each individual publication or patent application were specifically and individually indicated to be so incorporated by reference. Although the present invention has been described in some detail by way of illustration and example for purposes of clarity and understanding, it will be apparent that certain changes and modifications may be practiced within the scope of the appended claims.

What is claimed is:

1. A computerized method for determining whether a biological sequence has certain characteristic comprising:
obtaining a plurality of evidence about the characteristic, wherein at least one evidence is sequence annotation; and
determining the characteristic using a Bayesian analysis of the evidence.

2. The method of claim 1 wherein the step of determining comprises:
defining the prior probability of the biological sequence having the characteristic;
estimating the probability of the evidence assuming the hypothesis is true; and
calculating the probability that the hypothesis is true.

3. The method of claim 2 wherein the step of calculating is performed according to Bayes' rule.

4. The method of claim 3 wherein the biological sequence is a nucleic acid sequence and the characteristic is the orientation of the biological sequence.

5. The method of claim 4 wherein the nucleic acid sequence represents a cluster of nucleic acid sequences including at least one EST sequence.

6. The method of claim 5 wherein the plurality of evidence comprises evidence from poly-A/T tail analysis, inferred splice sites; and external sequence annotation.

7. The method of claim 6 wherein the external sequence annotation comprises RNA label and EST label.

8. The method of claim 7 further comprising testing a null hypothesis that the orientation determination is correct and conflicting evidence observed is due to random error.

9. A computerized method for designing nucleic acid probe arrays comprising:
obtaining a plurality of evidence about at least one characteristic of a target nucleic acid sequence, wherein at least one evidence is sequence annotation;
determining the characteristic using a Bayesian analysis of the evidence; and
defining a target region based upon the characteristic; and
selecting probes against the target region.

10. The method of claim 9 wherein the step of determining comprises defining the prior probability that a hypothesis that the target nucleic acid sequence has the characteristic; estimating the probability of the evidence assuming the hypothesis is true; and
calculating the probability that the hypothesis is true.

11. The method of claim 10 wherein the step of calculating is performed according to Bayes' Rule.

12. The method of claim 11 wherein the characteristic is the orientation of the target nucleic acid sequence.

13. The method of claim 12 wherein the nucleic acid sequence represents a cluster of nucleic acid sequences including at least one EST sequence.

14. The method of claim 13 wherein the plurality of evidence comprises evidence from poly-A/T tail analysis, inferred splice sites; and external sequence annotation.

15. The method of claim 14 wherein the external sequence annotation comprises RNA label and EST label.

16. The method of claim 15 further comprising testing a null hypothesis that the orientation determination is correct and conflicting evidence observed is due to random error.

17. A system for characterizing a biological sequence comprising a processor; and a memory coupled with the processor, the memory storing a plurality of machine instructions that cause the processor to perform logical steps of:
obtaining a plurality of evidence about the characteristic, wherein at least one evidence is sequence annotation; and
determining the characteristic using a Bayesian analysis of the evidence.

18. The system of claim 17 wherein the step of determining comprises:
defining the prior probability of the biological sequence having the characteristic;
estimating the probability of the evidence assuming the hypothesis is true; and
calculating the probability that the hypothesis is true.

19. The system of claim 18 wherein the step of calculating is performed according to Bayes' Rule.

20. The system of claim 19 wherein the biological sequence is a nucleic acid sequence and the characteristic is the orientation of the biological sequence.

21. The system of claim 20 wherein the nucleic acid sequence represents a cluster of nucleic acid sequences including at least one EST sequence.

22. The system of claim 22 wherein the plurality of evidence comprises evidence from poly-A/T tail analysis, inferred splice sites; and external sequence annotation.

23. The system of claim 22 wherein the external sequence annotation comprises RNA label and EST label.

24. The system of claim 23 further comprising testing a null hypothesis that the orientation determination is correct and conflicting evidence observed is due to random error.

25. A system for characterizing a biological sequence comprises a processor; and a memory coupled with the processor, the memory storing a plurality of machine instructions that cause the processor to perform logical steps of:
obtaining a plurality of evidence about at least one characteristic of a target nucleic acid sequence, wherein at least one evidence is sequence annotation;
determining the characteristic using a Bayesian analysis of the evidence; and
defining a target region based upon the characteristic; and
selecting probes against the target region.

26. The system of claim 25 wherein the step of determining comprises defining the prior probability that a hypothesis that the target nucleic acid sequence has the characteristic; estimating the probability of the evidence assuming the hypothesis is true; and
calculating the probability that the hypothesis is true.

27. The system of claim 26 wherein the step of calculating is performed according to Bayes' Rule.

28. The system of claim 27 wherein the characteristic is the orientation of the target nucleic acid sequence.

29. The system of claim 28 wherein the nucleic acid sequence represents a cluster of nucleic acid sequences including at least one EST sequence.

30. The system of claim 29 wherein the plurality of evidence comprises evidence from poly-A/T tail analysis, inferred splice sites; and external sequence annotation.

31. The system of claim 30 wherein the external sequence annotation comprises RNA label and EST label.

32. The system of claim 31 further comprising testing a null hypothesis that the orientation determination is correct and conflicting evidence observed is due to random error.
